(19) United States
(12) Patent Application Publication
(10) Pub. No.: US 2015/0313833 A1
(43) Pub. Date: Nov. 5, 2015

(54) METHOD OF DELIVERING SMOOTH FEEL TO HAIR

(71) Applicant: The Procter & Gamble Company, Cincinnati, OH (US)

(72) Inventors: Jennifer Elaine Hilvert, Cincinnati, OH (US); Denise Christine Winstel, Crescent Springs, KY (US)

(21) Appl. No.: 14/699,307

(22) Filed: Apr. 29, 2015

Related U.S. Application Data
(60) Provisional application No. 61/986,298, filed on Apr. 30, 2014.

(51) Int. Cl. A61K 8/898; A61Q 5/00; A61Q 5/02; A61K 8/891; A61K 8/06; A61K 8/04; A61K 8/41; A61K 8/73; A61Q 5/12

(52) U.S. Cl. CPC A61K 8/898; A61K 8/737; A61Q 5/006; A61Q 5/02; A61Q 5/12; A61K 8/06; A61K 8/042; A61K 8/416; A61K 8/891; A61K 2800/413

(57) ABSTRACT

The method of delivering a smooth hair feel described herein comprises applying to the hair a shampoo composition followed by a conditioner composition. The shampoo composition delivers consumer desired shampooing in addition to providing a smooth feel of the hair. The shampoo and conditioner compositions can be applied to the hair and rinsed off with water. When used together, such as part of a hair care regimen, the shampoo and conditioner compositions combinations deliver smooth feel of the hair.

### FIELD OF THE INVENTION

The present invention relates to a method of providing improved smooth hair feel via a shampoo and conditioner hair care regimen.

### BACKGROUND OF THE INVENTION

Shampoo and conditioner compositions comprising various combinations of detersive surfactants, conditioning agents, and carriers are known. These products typically include a shampoo which comprises an anionic detersive surfactant in combination with a cationic deposition polymer and a conditioning agent such as a silicone, a hydrocarbon oil, a fatty ester, or combinations thereof, and a conditioner composition comprising a cationic surfactant and silicone materials. These products have become more popular among consumers as a means of conveniently obtaining hair conditioning and cleansing performance.

Historically, it has been difficult to provide a rinse off hair care shampoo and conditioner regimen which delivers hair with a smooth feel without resulting in negative hair feel tradeoffs, such as greasy or over conditioning. In rinse off compositions a number of attempts have been made to deliver smooth feeling hair with limited success.

Accordingly, there is a need for an improved hair care regimen that improves the smooth feel of hair, but still delivers the consumer desirable cleaning and conditioning benefits.

### SUMMARY OF THE INVENTION

A method of providing a smooth feel to hair comprising: first applying to the hair a shampoo composition comprising: from about 2% to about 50% of one or more detersive surfactants, by weight of the shampoo composition; from about 0.1 to about 0.25 by weight, of a cationic guar deposition polymer having a molecular weight of from about 100,000 g/mol to about 2.2 million g/mol; from about 0.1 to about 1.5 by weight, of a silicone emulsion comprising an insoluble polysiloxane having a general formula of R¹—[O—SiR₂]ₙ—OR¹, wherein n is an integer, R is a substituted or unsubstituted C₁ to C₁₀ alkyl or aryl, and R¹ is a hydrogen or a substituted or unsubstituted C₁ to C₁₀ alkyl or aryl, wherein the insoluble polysiloxane has a molecular weight within the range from about 50,000 to about 500,000 g/mol, and an average particle size within the range from about 30 nm to about 10 μm, wherein a total content of a cyclic polysiloxane having a general formula:

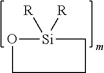

is present in the silicone emulsion in an amount less than about 2.5 wt % based on the total weight of the insoluble polysiloxane and the cyclic polysiloxane, wherein R is as defined above, wherein m is 4 or 5, from about 20% to about 95% of a first aqueous carrier, by weight of the shampoo composition; then rinsing the shampoo composition from the hair; nex applying to the hair a conditioner composition comprising: a conditioner gel matrix comprising: from about 0.1% to about 20% of one or more high melting point fatty compounds, by weight of the gel matrix; from about 0.1% to about 10% of a cationic surfactant, by weight of the gel matrix; wherein the cationic surfactant system comprises a mono-long alkyl quaternized ammonium salt at least 20% of a second aqueous carrier, by weight of the gel matrix; silicone conditioning agents and finally rinsing the conditioner composition from the hair.

### DETAILED DESCRIPTION OF THE INVENTION

While the specification concludes with claims particularly pointing out and distinctly claiming the invention, it is believed that the present invention will be better understood from the following description.

As used herein, the term “fluid” includes liquids and gels.

As used herein, the articles including “a” and “an” when used in a claim, are understood to mean one or more of what is claimed or described.

As used herein, “comprising” means that other steps and other ingredients which do not affect the end result can be added. This term encompasses the terms “consisting of” and “consisting essentially of”.

As used herein, “mixtures” is meant to include a simple combination of materials and any compounds that may result from their combination.

As used herein, “molecular weight” or “M.Wt.” refers to the weight average molecular weight unless otherwise stated. Molecular weight is measured using, gel permeation chromatography-LS (“GPC-LS”).

As used herein, the terms “include,” “includes,” and “including,” are meant to be non-limiting and are understood to mean “comprise,” “comprises,” and “comprising,” respectively.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

Unless otherwise noted, all component or composition levels are in reference to the active portion of that component or composition, and are exclusive of impurities, for example, residual solvents or by-products, which may be present in commercially available sources of such components or compositions.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### Shampoo/Conditioner Composition Regimen

The method of facilitating smooth hair feel described herein comprises applying to the hair a shampoo composition followed by a conditioner composition. The shampoo composition delivers consumer desired shampooing in addition delivering smooth feel to the hair.

After applying to the hair a shampoo composition as described herein, the method then comprises rinsing the shampoo composition from the hair and applying a rinse-off conditioner.

### Shampoo Composition

A. Detersive Surfactant

The shampoo composition may comprise one or more detersive surfactants, which provides cleaning performance to the composition. The one or more detersive surfactants in turn may comprise an anionic surfactant, amphoteric or zwitterionic surfactants, or mixtures thereof. Various examples and descriptions of detersive surfactants are set forth in U.S. Pat. No. 6,649,155; U.S. Patent Application Publication No. 2008/0317698; and U.S. Patent Application Publication No. 2008/0206355, which are incorporated herein by reference in their entirety.

The concentration of the detersive surfactant component in the shampoo composition should be sufficient to provide the desired cleaning and lather performance, and generally ranges from about 2 wt % to about 50 wt %, from about 5 wt % to about 30 wt %, from about 8 wt % to about 25 wt %, from about 10 wt % to about 20 wt %, about 5 wt %, about 10 wt %, about 12 wt %, about 15 wt %, about 17 wt %, about 18 wt %, or about 20 wt %.

Anionic surfactants suitable for use in the compositions are the alkyl and alkyl ether sulfates. Other suitable anionic surfactants are the water-soluble salts of organic, sulfuric acid reaction products. Still other suitable anionic surfactants are the reaction products of fatty acids esterified with isethionic acid and neutralized with sodium hydroxide. Other similar anionic surfactants are described in U.S. Pat. Nos. 2,486,921; 2,486,922; and 2,396,278, which are incorporated herein by reference in their entirety.

Exemplary anionic surfactants for use in the shampoo composition include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, sodium cocoyl isethionate and combinations thereof. In a further embodiment, the anionic surfactant is sodium lauryl sulfate or sodium laureth sulfate.

Suitable amphoteric or zwitterionic surfactants for use in the shampoo composition herein include those which are known for use in shampoo or other personal care cleansing. Concentrations of such amphoteric surfactants range from about 0.5 wt % to about 20 wt %, and from about 1 wt % to about 10 wt %. Non limiting examples of suitable zwitterionic or amphoteric surfactants are described in U.S. Pat. Nos. 5,104,646 and 5,106,609, which are incorporated herein by reference in their entirety.

Amphoteric detersive surfactants suitable for use in the shampoo composition include those surfactants broadly described as derivatives of aliphatic secondary and tertiary amines in which the aliphatic radical can be straight or branched chain and wherein one of the aliphatic substituents contains from about 8 to about 18 carbon atoms and one contains an anionic group such as carboxy, sulfonate, sulfate, phosphate, or phosphonate. Exemplary amphoteric detersive surfactants for use in the present shampoo composition include cocoamphoacetate, cocoamphodiacetate, lauroamphoacetate, lauroamphodiacetate, and mixtures thereof.

Zwitterionic detersive surfactants suitable for use in the shampoo composition include those surfactants broadly described as derivatives of aliphatic quaternaryammonium, phosphonium, and sulfonium compounds, in which the aliphatic radicals can be straight or branched chain, and wherein one of the aliphatic substituents contains from about 8 to about 18 carbon atoms and one contains an anionic group such as carboxy, sulfonate, sulfate, phosphate or phosphonate. In another embodiment, zwitterionics such as betaines are selected.

Non limiting examples of other anionic, zwitterionic, amphoteric or optional additional surfactants suitable for use in the shampoo composition are described in McCutcheon's, Emulsifiers and Detergents, 1989 Annual, published by M. C. Publishing Co., and U.S. Pat. Nos. 3,929,678, 2,658,072; 2,438,091; 2,528,378, which are incorporated herein by reference in their entirety.

The shampoo composition may also comprise a silicone, cationic deposition aid, an aqueous carrier, and other additional ingredients described herein.

B. Cationic Deposition Polymer

The shampoo composition also comprises a cationic deposition polymer. These cationic deposition polymers can include at least one cationic guar polymer. Additionally, the cationic deposition polymer can be a mixture of deposition polymers.

(1) Cationic Guar Polymers

According to an embodiment of the present invention, the shampoo composition comprises a cationic guar polymer, which is a cationically substituted galactomannan (guar) gum derivatives. Guar gum for use in preparing these guar gum derivatives is typically obtained as a naturally occurring material from the seeds of the guar plant. The guar molecule itself is a straight chain mannan, which is branched at regular intervals with single membered galactose units on alternative mannose units. The mannose units are linked to each other by means of β(1-4) glycosidic linkages. The galactose branching arises by way of an α(1-6) linkage. Cationic derivatives of the guar gums are obtained by reaction between the hydroxyl groups of the polygalactomannan and reactive quaternary ammonium compounds. The degree of substitution of the cationic groups onto the guar structure must be sufficient to provide the requisite cationic charge density described below.

According to one embodiment, the cationic guar polymer has a weight average M.Wt. of less than about 2.5 million g/mol, and has a charge density of from about 0.05 meq/g to about 2.5 meq/g. In an embodiment, the cationic guar polymer has a weight average M.Wt. of less than 1.5 million g/mol, or from about 150 thousand to about 1.5 million g/mol, or from about 200 thousand to about 1.5 million g/mol, or from about 300 thousand to about 1.5 million g/mol, or from about 700,000 thousand to about 1.5 million g/mol. In one embodiment, the cationic guar polymer has a charge density of from about 0.2 to about 2.2 meq/g, or from about 0.3 to about 2.0 meq/g, or from about 0.4 to about 1.8 meq/g; or from about 0.5 meq/g to about 1.7 meq/g.

According to one embodiment, the cationic guar polymer has a weight average M.Wt. of less than about 2.2 million g/mol, and has a charge density of from about 0.1 meq/g to about 2.5 meq/g. In an embodiment, the cationic guar polymer has a weight average M.Wt. of less than 2 million g/mol, or from about 150 thousand to about 1.7 million g/mol, or from about 200 thousand to about 700 thousand g/mol, or from about 300 thousand to about 700 thousand g/mol, or from about 400 thousand to about 600 thousand g/mol. from about 150 thousand to about 800 thousand g/mol, or from about 200 thousand to about 700 thousand g/mol, or from about 300 thousand to about 700 thousand g/mol, or from about 400 thousand to about 600 thousand g/mol. In one embodiment, the cationic guar polymer has a charge density of from about 0.2 to about 2.2 meq/g, or from about 0.3 to about 2.0 meq/g, or from about 0.4 to about 1.8 meq/g; or from about 0.5 meq/g to about 1.5 meq/g. The shampoo composition may also contain a blend of guars having varying molecular weights. In one embodiment the shampoo composition comprises a blend of guar having a molecular weight of about 500,000 g/mol and a guar having a molecular weight of about 1.7 million g/mol. The guar blends can have a ratio of from about 2:1 to about 1:2. In another embodiment the guar blends can have a ratio of about 1:1.

In an embodiment, the composition comprises from about 0.01% to less than about 0.7%, or from about 0.04% to about 0.55%, or from about 0.08% to about 0.5%, or from about 0.16% to about 0.5%, or from about 0.2% to about 0.5%, or from about 0.3% to about 0.5%, or from about 0.4% to about 0.5%, of cationic guar polymer (a), by total weight of the composition.

The cationic guar polymer may be formed from quaternary ammonium compounds. In an embodiment, the quaternary ammonium compounds for forming the cationic guar polymer conform to the general formula 1:

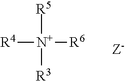

wherein where R³, R⁴ and R⁵ are methyl or ethyl groups; R⁶ is either an epoxyalkyl group of the general formula 2:

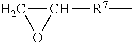

or R⁶ is a halohydrin group of the general formula 3:

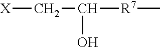

wherein R⁷ is a C₁ to C₃ alkylene; X is chlorine or bromine, and Z is an anion such as Cl—, Br—, I— or HSO₄—.

In an embodiment, the cationic guar polymer conforms to the general formula 4:

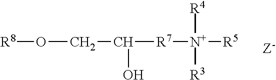

wherein R⁸ is guar gum; and wherein R⁴, R⁵, R⁶ and R⁷ are as defined above; and wherein Z is a halogen. In an embodiment, the cationic guar polymer conforms to Formula 5:

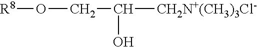

Suitable cationic guar polymers include cationic guar gum derivatives, such as guar hydroxypropyltrimonium chloride. In an embodiment, the cationic guar polymer is a guar hydroxypropyltrimonium chloride. Specific examples of guar hydroxypropyltrimonium chlorides include the Jaguar® series commercially available from Rhone-Poulenc Incorporated, for example Jaguar® C-500, commercially available from Rhodia. Jaguar® C-500 has a charge density of 0.8 meq/g and a M.Wt. of 500,000 g/mole. Jaguar® C-17, which has a cationic charge density of about 0.6 meq/g and a M.Wt. of about 2.2 million g/mol and is available from Rhodia Company. Jaguar® C 13S which has a M.Wt. of 2.2 million g/mol and a cationic charge density of about 0.8 meq/g (available from Rhodia Company). Other suitable guar hydroxypropyltrimonium chloride are: guar hydroxypropyltrimonium chloride which has a charge density of about 1.1 meq/g and a M.Wt. of about 500,000 g/mole is available from ASI, a charge density of about 1.5 meq/g and a M.Wt. of about 500,000 g/mole is available from ASI. Other suitable guar hydroxypropyltrimonium chloride are: Hi-Care 1000, which has a charge density of about 0.7 meq/g and a M.Wt. of about 600,000 g/mole and is available from Rhodia; N-Hance 3269 and N-Hance 3270, which has a charge density of about 0.7 meq/g and a M.Wt. of about 425,000 g/mole and is available from ASI; N-Hance 3196, which has a charge density of about 0.8 and a M. Wt. Of about 1,700,000 g/mole and is available from ASI. AquaCat CG518 has a charge density of about 0.9 meq/g and a M.Wt. of about 50,000 g/mole and is available from ASI. BF-13, which is a borate (boron) free guar of charge density of about 1.1 meq/g and M. W.t of about 800,000 and BF-17, which is a borate (boron) free guar of charge density of about 1.7 meq/g and M. W.t of about 800,000 both available from ASI.

C. Silicone Emulsion

The silicone emulsions suitable for use in the embodiments of the present invention include emulsions of insoluble polysiloxanes prepared in accordance with the descriptions provided in U.S. Pat. No. 4,476,282 and U.S. Patent Application Publication No. 2007/0276087. Accordingly, insoluble polysiloxanes referred to herein for the purpose of the invention include polysiloxanes such as alpha, omega hydroxy-terminated polysiloxanes or alpha, omega alkoxy-terminated polysiloxanes having a molecular weight within the range from about 50,000 to about 500,000 g/mol. As used herein, “insoluble polysiloxane” means that the water solubility of the polysiloxane is less than 0.05 wt %. In another embodiment, the water solubility of the polysiloxane is less than 0.02 wt %, or less than 0.01 wt %, or less than 0.001 wt %. According to an embodiment, the insoluble polysiloxane is present in the personal care composition in an amount within the range from about 0.1 wt % to about 3 wt %, based on the total weight of the composition. For example, the insoluble polysiloxane can be present in an amount within the range from about 0.1 wt % to about 1.75 wt %, or from about 0.2% to about 1.75%, or from about 0.4 wt % to about 1.5 wt %, or from about 0.5 wt % to about 1.5 wt %, based on the total weight of the composition.

According to one aspect of the silicone emulsion, the insoluble polysiloxane used herein include alpha, omega hydroxy- or alkoxy-terminated polysiloxanes having a general formula I:

R1-[O—SiR2]n-OR1,

wherein ‘n’ is an integer, R is a substituted or unsubstituted C1 to C10 alkyl or aryl, and R1 is a hydrogen or a substituted or unsubstituted C1 to C10 alkyl or aryl. Non-limiting examples of R and R1 may be independently selected from alkyl groups such as methyl, ethyl, n-propyl, isopropyl, n-butyl, isobutyl, tert-butyl, n-pentyl, isopentyl, neopentyl, tertpentyl, hexyl such as n-hexyl, heptyl such as n-heptyl, octyl such as n-octyl and isooctyl such as 2,2,4-trimethyl-pentyl, nonyl such as n-nonyl, decyl such as n-decyl, dodecyl such as n-dodecyl, octadecyl such as n-octadecyl; or aryl groups such as phenyl, naphthyl, anthryl and phenanthryl. In an embodiment, the insoluble polysiloxane has a general formula H—[O—SiR2]n-OH.

According to another aspect of the silicone emulsion, the insoluble polysiloxane has a molecular weight within the range from about 50,000 to about 500,000 g/mol. For example, the insoluble polysiloxane may have a molecular weight within the range from about 60,000 to about 400,000; from about 75,000 to about 300,000; from about 100,000 to about 200,000; or the molecular weight may be about 150,000 g/mol.

According to another aspect of the silicon emulsion, total content of a cyclic polysiloxane having a general formula:

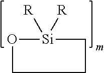

wherein R is as defined above, and wherein m is 4 or 5, is present in the silicone emulsion in an amount less than about 2.5 wt % based on the total weight of all polysiloxanes. For example, dimethiconol may include significant quantities of cyclic polysiloxanes, such as octamethylcyclotetrasiloxane (D4) and decamethylcyclotetrasiloxane (D5). In an embodiment, the amount of D4 is less than about 2.0%, or less than about 1.5%, or less than about 1.0%, or less than about 0.5%, based on the total weight of all polysiloxanes. In an embodiment, the amount of D5 is less than about 0.5%, or less than about 0.4%, or less than about 0.3%, or less than about 0.2%, based on the total weight of all polysiloxanes.

According to yet another aspect of the silicone emulsion, the emulsion has a viscosity up to about 500,000 cPs. For example, the viscosity may be within the range from about 75,000 to about 300,000, from about 100,000 to about 200,000, or about 150,000 cPs.

According to yet another aspect of the silicone emulsion, the insoluble polysiloxane has an average particle size within the range from about 30 nm to about 10 micron. The average particle size may be within the range from about 40 nm to about 5 micron, from about 50 nm to about 1 micron, from about 75 nm to about 500 nm, or about 100 nm, for example.

The molecular weight of the insoluble polysiloxane, the viscosity of the silicone emulsion, and the size of the particle comprising the insoluble polysiloxane are determined by methods commonly used by those skilled in the art, such as the methods disclosed in Smith, A. L. The Analytical Chemistry of Silicones, John Wiley & Sons, Inc.: New York, 1991. For example, the viscosity of the silicone emulsion can be measured at 30° C. with a Brookfield viscosimeter with spindle 6 at 2.5 rpm.

According to another aspect of the silicone emulsion, the emulsion further includes an anionic surfactant that participates in providing high internal phase viscosity emulsions having particle sizes in the range from about 30 nm to about 10 micron. The anionic surfactant is selected from organic sulfonic acids. Most common sulfonic acids used in the present process are alkylaryl sulfonic acid; alkylaryl polyoxyethylene sulphonic acid; alkyl sulfonic acid; and alkyl polyoxyethylene sulfonic acid. General formulas of the sulfonic acids are as shown below:

R2C6H4SO3H (II)

R2C6H4O(C2H4O)mSO3H (III)

R2SO3H (IV)

R2O(C2H4O)mSO3H (IV)

Where R2, which may differ, is a monovalent hydrocarbon radical having at least 6 carbon atoms. Non-limiting examples of R2 include hexyl, octyl, decyl, dodecyl, cetyl, stearyl, myristyl, and oleyl. ‘m’ is an integer from 1 to 25. Exemplary anionic surfactants include but are not limited to octylbenzene sulfonic acid; dodecylbenzene sulfonic acid; cetylbenzene sulfonic acid; alpha-octyl sulfonic acid; alpha-dodecyl sulfonic acid; alpha-cetyl sulfonic acid; polyoxyethylene octylbenzene sulfonic acid; polyoxyethylene dodecylbenzene sulfonic acid; polyoxyethylene cetylbenzene sulfonic acid; polyoxyethylene octyl sulfonic acid; polyoxyethylene dodecyl sulfonic acid; and polyoxyethylene cetyl sulfonic acid. Generally, 1 to 15% anionic surfactant is used in the emulsion process. For example, 3-10% anionic surfactant can be used to obtain an optimum result.

The silicone emulsion may further include an additional emulsifier together with the anionic surfactant, which along with the controlled temperature of emulsification and polymerization, facilitates making the emulsion in a simple and faster way. Non-ionic emulsifiers having a hydrophilic lipophilic balance (HLB) value of 10 to 19 are suitable and include polyoxyalkylene alkyl ether, polyoxyalkylene alkylphenyl ethers and polyoxyalkylene sorbitan esters. Some useful emulsifiers having an HLB value of 10 to 19 include, but are not limited to, polyethylene glycol octyl ether; polyethylene glycol lauryl ether; polyethylene glycol tridecyl ether; polyethylene glycol cetyl ether; polyethylene glycol stearyl ether; polyethylene glycol nonylphenyl ether; polyethylene glycol dodecylphenyl ether; polyethylene glycol cetylphenyl ether; polyethylene glycol stearylphenyl ether; polyethylene glycol sorbitan mono stearate; and polyethylene glycol sorbitan mono oleate.

D. Aqueous Carrier

The shampoo composition comprises a first aqueous carrier. Accordingly, the formulations of the shampoo composition can be in the form of pourable liquids (under ambient conditions). Such compositions will therefore typically comprise a first aqueous carrier, which is present at a level of at least 20 wt %, from about 20 wt % to about 95 wt %, or from about 60 wt % to about 85 wt %. The first aqueous carrier may comprise water, or a miscible mixture of water and organic solvent, and in one aspect may comprise water with minimal or no significant concentrations of organic solvent, except as otherwise incidentally incorporated into the composition as minor ingredients of other components.

The first aqueous carriers useful in the shampoo composition include water and water solutions of lower alkyl alcohols and polyhydric alcohols. The lower alkyl alcohols useful herein are monohydric alcohols having 1 to 6 carbons, in one aspect, ethanol and isopropanol. The polyhydric alcohols useful herein include propylene glycol, hexylene glycol, glycerin, and propane diol.

### Conditioner Composition

The method of facilitating smooth feel of hair described herein comprises applying to the hair a conditioner composition after rinsing the shampoo composition from the hair. The conditioner composition described herein delivers consumer desired conditioning in addition to delivering smooth hair feel.

The conditioner composition described herein comprises (i) from about 0.1 to about 1.5% silicone fluid (ii) a conditioner gel matrix. The conditioner gel matrix comprises (1) one or more high melting point fatty compounds, (2) a cationic surfactant, and (3) a second aqueous carrier. After applying to the hair a conditioner composition as described herein, the method then comprises rinsing the conditioner composition from the hair.

A. Cationic Surfactant

The composition of the present invention comprises a cationic surfactant. The cationic surfactant system can be selected from a mono-long alkyl quaternized ammonium salt;

The cationic surfactant can be included in the composition at a level by weight of from about 0.1% to about 10%, from about 0.5% to about 8%, from about 0.8% to about 5%, and from about 1.0% to about 4%.

Suitable cationic surfactants comprise mono-long alkyl quaternized ammonium salt alone without additional cationic surfactants added at efficacious levels (efficacious levels include levels above 0.25 by weight).

### Mono-Long Alkyl Quaternized Ammonium Salt

The monoalkyl quaternized ammonium salt cationic surfactants useful herein are those having one long alkyl chain which has from 12 to 30 carbon atoms, from 16 to 24 carbon atoms, and in one embodiment at C18-22 alkyl group. The remaining groups attached to nitrogen are independently selected from an alkyl group of from 1 to about 4 carbon atoms or an alkoxy, polyoxyalkylene, alkylamido, hydroxyalkyl, aryl or alkylaryl group having up to about 4 carbon atoms.

Mono-long alkyl quaternized ammonium salts useful herein are those having the formula (I):

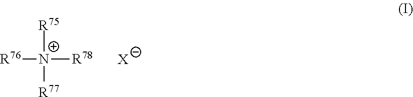

wherein one of R⁷⁵, R⁷⁶, R⁷⁷ and R⁷⁸ is selected from an alkyl group of from 12 to 30 carbon atoms or an aromatic, alkoxy, polyoxyalkylene, alkylamido, hydroxyalkyl, aryl or alkylaryl group having up to about 30 carbon atoms; the remainder of R⁷⁵, R⁷⁶, R⁷⁷ and R⁷⁸ are independently selected from an alkyl group of from 1 to about 4 carbon atoms or an alkoxy, polyoxyalkylene, alkylamido, hydroxyalkyl, aryl or alkylaryl group having up to about 4 carbon atoms; and X⁻ is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The alkyl groups can contain, in addition to carbon and hydrogen atoms, ether and/or ester linkages, and other groups such as amino groups. The longer chain alkyl groups, e.g., those of about 12 carbons, or higher, can be saturated or unsaturated. One of R⁷⁵, R⁷⁶, R⁷⁷ and R⁷⁸ can be selected from an alkyl group of from 12 to 30 carbon atoms, from 16 to 24 carbon atoms, from 18 to 22 carbon atoms, an/or 22 carbon atoms; the remainder of R⁷⁵, R⁷⁶, R⁷⁷ and R⁷⁸ are independently selected from CH₃, C₂H₅, C₂H₄OH, and mixtures thereof; and X is selected from the group consisting of Cl, Br, CH₃OSO₃, C₂H₅OSO₃, and mixtures thereof.
Nonlimiting examples of such mono-long alkyl quaternized ammonium salt cationic surfactants include: behenyl trimethyl ammonium salt; stearyl trimethyl ammonium salt; cetyl trimethyl ammonium salt; and hydrogenated tallow alkyl trimethyl ammonium salt.

B. High Melting Point Fatty Compound

The high melting point fatty compound useful herein have a melting point of 25° C. or higher, and is selected from the group consisting of fatty alcohols, fatty acids, fatty alcohol derivatives, fatty acid derivatives, and mixtures thereof. It is understood by the artisan that the compounds disclosed in this section of the specification can in some instances fall into more than one classification, e.g., some fatty alcohol derivatives can also be classified as fatty acid derivatives. However, a given classification is not intended to be a limitation on that particular compound, but is done so for convenience of classification and nomenclature. Further, it is understood by the artisan that, depending on the number and position of double bonds, and length and position of the branches, certain compounds having certain required carbon atoms may have a melting point of less than 25° C. Such compounds of low melting point are not intended to be included in this section. Nonlimiting examples of the high melting point compounds are found in International Cosmetic Ingredient Dictionary, Fifth Edition, 1993, and CTFA Cosmetic Ingredient Handbook, Second Edition, 1992.

Among a variety of high melting point fatty compounds, fatty alcohols are suitable for use in the composition of the present invention. The fatty alcohols useful herein are those having from about 14 to about 30 carbon atoms, from about 16 to about 22 carbon atoms. These fatty alcohols are saturated and can be straight or branched chain alcohols. Suitable fatty alcohols include, for example, cetyl alcohol, stearyl alcohol, behenyl alcohol, and mixtures thereof.

High melting point fatty compounds of a single compound of high purity can be used. Single compounds of pure fatty alcohols selected from the group of pure cetyl alcohol, stearyl alcohol, and behenyl alcohol can also be used. By “pure” herein, what is meant is that the compound has a purity of at least about 90%, and/or at least about 95%. These single compounds of high purity provide good rinsability from the hair when the consumer rinses off the composition.

The high melting point fatty compound is included in the composition at a level of from about 0.1% to about 20%, from about 1% to about 15%, and/or from about 1.5% to about 8% by weight of the composition, in view of providing improved conditioning benefits such as slippery feel during the application to wet hair, softness and moisturized feel on dry hair.

### C. Aqueous Carrier

The gel matrix of the hair care composition of the present invention includes an aqueous carrier. Accordingly, the formulations of the present invention can be in the form of pourable liquids (under ambient conditions). Such compositions will therefore typically comprise an aqueous carrier, which is present at a level of from about 20 wt % to about 95 wt %, or from about 60 wt % to about 85 wt %. The aqueous carrier may comprise water, or a miscible mixture of water and organic solvent, and in one aspect may comprise water with minimal or no significant concentrations of organic solvent, except as otherwise incidentally incorporated into the composition as minor ingredients of other components.

The aqueous carrier useful in the present invention includes water and water solutions of lower alkyl alcohols and polyhydric alcohols. The lower alkyl alcohols useful herein are monohydric alcohols having 1 to 6 carbons, in one aspect, ethanol and isopropanol. The polyhydric alcohols useful herein include propylene glycol, hexylene glycol, glycerin, and propane diol.

According to embodiments of the present invention, the hair care compositions may have a pH in the range from about 2 to about 10, at 25° C. In one embodiment, the hair care composition has a pH in the range from about 2 to about 6, which may help to solubilize minerals and redox metals already deposited on the hair. Thus, the hair care composition can also be effective toward washing out the existing minerals and redox metals deposits, which can reduce cuticle distortion and thereby reduce cuticle chipping and damage.

### D. Gel Matrix

The composition of the present invention comprises a gel matrix. The gel matrix comprises a cationic surfactant, a high melting point fatty compound, and an aqueous carrier. The gel matrix is suitable for providing various conditioning benefits such as slippery feel during the application to wet hair and softness and moisturized feel on dry hair. In view of providing the above gel matrix, the cationic surfactant and the high melting point fatty compound are contained at a level such that the weight ratio of the cationic surfactant to the high melting point fatty compound is in the range of, from about 1:1 to about 1:10, and/or from about 1:1 to about 1:6.

### E. Additional Components

1. Silicone Conditioning Agent

According to embodiments of the present invention, the hair care composition includes a silicone conditioning agent which comprises a silicone compound. The silicone compound may comprise volatile silicone, non-volatile silicones, or combinations thereof. In one aspect, non-volatile silicones are employed. If volatile silicones are present, it will typically be incidental to their use as a solvent or carrier for commercially available forms of non-volatile silicone materials ingredients, such as silicone gums and resins. The silicone compounds may comprise a silicone fluid conditioning agent and may also comprise other ingredients, such as a silicone resin to improve silicone fluid deposition efficiency or enhance glossiness of the hair. The concentration of the silicone compound in the conditioner composition typically ranges from about 0.01 wt % to about 10 wt %, from about 0.1 wt % to about 8 wt %, from about 0.1 wt % to about 5 wt %, or even from about 0.2 wt % to about 3 wt %, for example

Exemplary silicone compounds include (a) a first polysiloxane which is non-volatile, substantially free of amino groups, and has a viscosity of from about 100,000 mm²s⁻¹ to about 30,000,000 mm²s⁻¹; (b) a second polysiloxane which is non-volatile, substantially free of amino groups, and has a viscosity of from about 5 mm²s⁻¹ to about 10,000 mm²s⁻¹; (c) an aminosilicone having less than about 0.5 wt % nitrogen by weight of the aminosilicone; (d) a silicone copolymer emulsion with an internal phase viscosity of greater than about 100×10⁶ mm²s⁻¹, as measured at 25° C.; (e) a silicone polymer containing quaternary groups; or (f) a grafted silicone polyol, wherein the silicone compounds (a)-(f) are disclosed in U.S. Patent Application Publication Nos. 2008/0292574, 2007/0041929, 2008/0292575, and 2007/0286837, each of which is incorporated by reference herein in its entirety.

c. Aminosilicone

The hair care composition of the present invention may comprise an amino silicone having less than about 0.5 wt % nitrogen by weight of the aminosilicone, such as less than about 0.2 wt %, or less than about 0.1 wt %, in view of friction reduction benefit. It has been surprisingly found that higher levels of nitrogen (amine functional groups) in the amino silicone tend to result in less friction reduction, and consequently less conditioning benefit from the aminosilicone. The aminosilicone useful herein may have at least one silicone block with greater than 200 siloxane units, in view of friction reduction benefit. The aminosilicones useful herein include, for example, quaternized aminosilicone and non-quaternized aminosilicone.

In one embodiment, the aminosilicones useful herein are water-insoluble. In the present invention, “water-insoluble aminosilicone” means that the aminosilicone has a solubility of 10 g or less per 100 g water at 25° C., in another embodiment 5 g or less per 100 g water at 25° C., and in another embodiment 1 g or less per 100 g water at 25° C. In the present invention, “water-insoluble aminosilicone” means that the aminosilicone is substantially free of copolyol groups. If copolyol groups are present, they are present at a level of less than 10 wt %, less than 1 wt %, or less than 0.1 wt % by weight of the aminosilicone.

According to one embodiment, aminosilicone useful herein are those which conform to the general formula (III):

(R²)ₐG₃₋ₐ-Si(—O—SiG₂)ₙ(—O—SiG_{b}(R²)_{2-b})ₘ—O—SiG₃₋ₐ(R²)ₐ (IIII)

wherein G is hydrogen, phenyl, hydroxy, or C₁-C₈ alkyl, such as methyl; a is an integer having a value from 1 to 3, such as 1; b is an integer having a value from 0 to 2, such as 1; n is a number from 1 to 2,000, such as from 100 to 1,800, from 300 to 800, or from 500 to 600; m is an integer having a value from 0 to 1,999, such as from 0 to 10, or 0; R² is a monovalent radical conforming to the general formula C_{q}H_{2q}L, wherein q is an integer having a value from 2 to 8 and L is selected from the following groups: —N(R³₂)CH₂—CH₂—N(R³₂)₂; —N(R³)₂; —N⁺(R³)₃A⁻; —N(R³)CH₂—CH₂—N⁺R³H₂A⁻; wherein R³ is hydrogen, phenyl, benzyl, or a saturated hydrocarbon radical, such as an alkyl radical from about C₁ to about C₂₀; A⁻ is a halide ion. According to an embodiment, L is —N(CH₃)₂ or —NH₂. According to another embodiment, L is —NH₂.

The aminosilicone of the above formula is used at levels by weight of the composition of from about 0.1 wt % to about 5 wt %, alternatively from about 0.2 wt % to about 2 wt %, alternatively from about 0.2 wt % to about 1.0 wt %, and alternatively from about 0.3 wt % to about 0.8 wt %.

According to one embodiment, the aminosilicone may include those compounds corresponding to formula (III) wherein m=0; a=1; q=3; G=methyl; n is from about 1400 to about 1700, such as about 1600; and L is —N(CH₃)₂ or —NH₂, such as —NH₂. According to another embodiment, the aminosilicone may include those compounds corresponding to formula (III) wherein m=0; a=1; q=3; G=methyl; n is from about 400 to about 800, such as from about 500 to around 600; and L is L is —N(CH₃)₂ or —NH₂, such as —NH₂. Accordingly, the aforementioned aminosilicones can be called terminal aminosilicones, as one or both ends of the silicone chain are terminated by nitrogen containing group. Such terminal aminosilicones may provide improved friction reduction compared to graft aminosilicones.

Another example of an aminosilicone useful herein includes, for example, quaternized aminosilicone having a tradename KF8020 available from Shinetsu.

The above aminosilicones, when incorporated into the hair care composition, can be mixed with solvent having a lower viscosity. Such solvents include, for example, polar or non-polar, volatile or non-volatile oils. Such oils include, for example, silicone oils, hydrocarbons, and esters. Among such a variety of solvents, exemplary solvents include those selected from the group consisting of non-polar, volatile hydrocarbons, volatile cyclic silicones, non-volatile linear silicones, and mixtures thereof. The non-volatile linear silicones useful herein are those having a viscosity of from about 1 mm²s⁻¹ to about 20,000 mm²s⁻¹, such as from about 20 mm²s⁻¹ to about 10,000 mm²s⁻¹, at 25° C. According to one embodiment, the solvents are non-polar, volatile hydrocarbons, especially non-polar, volatile isoparaffins, in view of reducing the viscosity of the aminosilicones and providing improved hair conditioning benefits such as reduced friction on dry hair. Such mixtures may have a viscosity of from about 1,000 mPa·s to about 100,000 mPa·s, and alternatively from about 5,000 mPa·s to about 50,000 mPa·s.

2. Other Conditioning Agents

Also suitable for use in the hair care compositions herein are the conditioning agents described by the Procter & Gamble Company in U.S. Pat. Nos. 5,674,478, and 5,750,122. Also suitable for use herein are those conditioning agents described in U.S. Pat. Nos. 4,529,586, 4,507,280, 4,663,158, 4,197,865, 4,217, 914, 4,381,919, and 4,422, 853.

3. Suspending Agent

The hair care compositions of the present invention may further comprise a suspending agent at concentrations effective for suspending water-insoluble material in dispersed form in the compositions or for modifying the viscosity of the composition. Such concentrations range from about 0.1 wt % to about 10 wt %, or even from about 0.3 wt % to about 5.0 wt %.

Optional suspending agents include crystalline suspending agents which can be categorized as acyl derivatives, long chain amine oxides, and mixtures thereof. These suspending agents are described in U.S. Pat. No. 4,741,855.

These suspending agents include ethylene glycol esters of fatty acids in one aspect having from about 16 to about 22 carbon atoms. In one aspect, useful suspending agents include ethylene glycol stearates, both mono and distearate, but in one aspect, the distearate containing less than about 7% of the mono stearate. Other suitable suspending agents include alkanol amides of fatty acids, having from about 16 to about 22 carbon atoms, or even about 16 to 18 carbon atoms, examples of which include stearic monoethanolamide, stearic diethanolamide, stearic monoisopropanolamide and stearic monoethanolamide stearate. Other long chain acyl derivatives include long chain esters of long chain fatty acids (e.g., stearyl stearate, cetyl palmitate, etc.); long chain esters of long chain alkanol amides (e.g., stearamide diethanolamide distearate, stearamide monoethanolamide stearate); and glyceryl esters (e.g., glyceryl distearate, trihydroxystearin, tribehenin) a commercial example of which is Thixin® R available from Rheox, Inc. Long chain acyl derivatives, ethylene glycol esters of long chain carboxylic acids, long chain amine oxides, and alkanol amides of long chain carboxylic acids in addition to the materials listed above may be used as suspending agents.

Other long chain acyl derivatives suitable for use as suspending agents include N,N-dihydrocarbyl amido benzoic acid and soluble salts thereof (e.g., Na, K), particularly N,N-di(hydrogenated) C16, C18 and tallow amido benzoic acid species of this family, which are commercially available from Stepan Company (Northfield, Ill., USA).

Examples of suitable long chain amine oxides for use as suspending agents include alkyl dimethyl amine oxides, e.g., stearyl dimethyl amine oxide.

Other suitable suspending agents include primary amines having a fatty alkyl moiety having at least about 16 carbon atoms, examples of which include palmitamine or stearamine, and secondary amines having two fatty alkyl moieties each having at least about 12 carbon atoms, examples of which include dipalmitoylamine or di(hydrogenated tallow)amine Still other suitable suspending agents include di(hydrogenated tallow)phthalic acid amide, and crosslinked maleic anhydride-methyl vinyl ether copolymer.

### Additional Hair Care Composition Components

The shampoo composition and/or conditioner composition (hair care compositions) described herein may optionally comprise one or more additional components known for use in hair care or personal care products, provided that the additional components are physically and chemically compatible with the essential components described herein, or do not otherwise unduly impair product stability, aesthetics or performance. Such additional components are most typically those described in reference books such as the CTFA Cosmetic Ingredient Handbook, Second Edition, The Cosmetic, Toiletries, and Fragrance Association, Inc. 1988, 1992. Individual concentrations of such additional components may range from about 0.001 wt % to about 10 wt % by weight of the hair care compositions.

### Hair Care Composition Forms

The hair care compositions of the present invention may be presented in typical hair care formulations. They may be in the form of solutions, dispersion, emulsions, powders, talcs, encapsulated, spheres, spongers, foams, and other delivery mechanisms. The compositions of the embodiments of the present invention may be hair tonics, leave-on hair products such as treatment, and styling products, rinse-off hair products such as shampoos, and treatment products; and any other form that may be applied to hair.

The hair care compositions are generally prepared by conventional methods such as are known in the art of making the compositions. Such methods typically involve mixing of the ingredients in one or more steps to a relatively uniform state, with or without heating, cooling, application of vacuum, and the like. The compositions are prepared such as to optimize stability (physical stability, chemical stability, photostability) and/or delivery of the active materials. The hair care composition may be in a single phase or a single product, or the hair care composition may be in a separate phases or separate products. If two products are used, the products may be used together, at the same time or sequentially. Sequential use may occur in a short period of time, such as immediately after the use of one product, or it may occur over a period of hours or days.

### EXAMPLES

The following examples illustrate embodiments of the invention described herein. The exemplified shampoo compositions, and/or conditioner compositions can be prepared by conventional formulation and mixing techniques. It will be appreciated that other modifications of the shampoo compositions, and/or conditioner compositions within the skill of those in the formulation art can be undertaken without departing from the spirit and scope of this invention. All parts, percentages, and ratios herein are by weight unless otherwise specified. Some components may come from suppliers as dilute solutions. The amount stated reflects the weight percent of the active material, unless otherwise specified.

The following are non-limiting examples of shampoo compositions, conditioner compositions, and leave-on treatments described herein.

### Shampoo Examples

### Non Limiting Examples

| Example | 1 | 2 | 3 | 4 | 5 | 6 | 7 |
|---|---|---|---|---|---|---|---|
| Sodium Lauryl Sulfate¹ | 7.0 | 7.0 | 7.0 | 7.0 | 0.00 | 7.0 | 7.0 |
| Sodium Laureth-3 Sulfate² | 8.0 | 8.0 | 8.0 | 8.0 | 0.0 | 6.0 | 8.0 |
| Sodium Laureth-1 Sulfate³ | 0 | 0.0 | 0.0 | 0.0 | 15.0 | 0.0 | 0 |
| Cocamidopropyl betaine⁴ | 2.0 | 2.0 | 2.0 | 1.50 | 1.75 | 2.0 | 2.0 |
| Cocomonoethanolamide⁵ | 0.0 | 0.0 | 0.0 | 0.85 | 0.0 | 0.0 | 0.0 |
| Ethylene Glycol Distearate | 1.5 | 1.50 | 1.50 | 1.50 | 1.50 | 0.75 | 1.5 |
| Dimethicone/Dimethiconol^{6.} | 0.8 | 1.0 | 1.0 | 0.75 | 0.8 | 0.8 | 1.0 |
| Fragrance | 0.8 | 0.8 | 0.8 | 0.8 | 0.8 | 0.6 | 0.8 |
| Guar Hydroxypropyltrimonium | 0.15 | 0.1 | 0.15 | 0.1 | 0.2 | 0.15 | 0.25 |
| chloride⁷ | | | | | | | |
| Guar Hydroxypropyltrimonium | 0.15 | 0.1 | 0.15 | 0.1 | 0.2 | 0.15 | 0.0 |
| chloride⁸ | | | | | | | |
| Methyl Paraben | 0.0 | 0.0 | 0.10 | 0.0 | 0.0 | 0.0 | 0.0 |
| Sodium Benzoate | 0.25 | 0.25 | 0.50 | 0.25 | 0.25 | 0.25 | 0.25 |
| Salicylic Acid USP | 0.0 | 0.0 | 0.20 | 0.0 | 0.0 | 0.0 | 0.0 |
| Tetrasodium EDTA | 0.15 | 0.15 | 0.15 | 0.15 | 0.15 | 0.15 | 0.15 |
| Tetrahydrate | | | | | | | |
| Trisodium Ethylenediamine | 0.10 | 0.10 | 0.20 | 0.10 | 0.10 | 0.10 | 0.1 |
| Disuccinate | | | | | | | |
| Panthenol | 0.03 | 0.03 | 0.03 | 0.03 | 0.03 | 0.03 | 0.03 |
| Panthenyl Ethyl Ether | 0.03 | 0.03 | 0.03 | 0.03 | 0.03 | 0.03 | 0.03 |
| Methylchloroisothiazolinone | 0.0005 | 0.0005 | 0.0 | 0.0005 | 0.0005 | 0.0005 | 0.0005 |

¹Stepanol SLS from Stepan
²P&G Chemicals
³P&G Chemicals
⁴Amphosol HCA-HP from Stepan
⁵Ninol Comf from Stepan
^{6.}Besil DM500 from Wacker
⁷N-Hance 3196 from Aqualon
⁸Jaguar C500 from Rhodia

### Conditioner Examples

| EXAMPLE | 1 | 2 | 3 | 4 |
|---|---|---|---|---|
| Stearyl alcohol | 4.64 | 4.65 | 4.75 | 4.5 |
| Behentrimonium chloride | 2.28 | 2.2 | 2.5 | 2.25 |
| Cetyl alcohol | 1.86 | 1.85 | 2.0 | 1.75 |
| Bis-aminopropyl dimethicone | 1.5 | 1.5 | 1.25 | 1.75 |
| Fragrance | 0.5 | 0.5 | 0.5 | 0.5 |
| Benzyl alcohol | 0.4 | 0.4 | 0.4 | 0.4 |
| Disodium edta | 0.1 | 0.1 | 0.1 | 0.1 |
| Panthenol | 0.03 | 0.03 | 0.03 | 0.03 |
| Panthenyl Ethyl Ether | 0.03 | 0.03 | 0.03 | 0.03 |
| Methylchloroisothiazolinone | 0.0005 | 0.0005 | 0.0005 | 0.0005 |

1. P&G Chemical
2. Incroquat Behenyl TMC-85-PA- Croda
3. P&G Chemical
4. Momentive

### Comparative Examples Shampoo A-C

| Example | | B |
|---|---|---|
| Sodium Lauryl Sulfate¹ | 7.0 | 7.0 |
| Sodium Laureth-3 Sulfate² | 8.0 | 8.0 |
| Cocamidopropyl betaine³ | 2.0 | 2.0 |
| Ethylene Glycol Distearate | 1.50 | 1.50 |
| Dimethicone/Dimethiconol⁴ | 0.5 | 2.0 |
| Fragrance | 0.8 | 0.8 |
| Guar Hydroxypropyltrimonium chloride⁵ | 0 | 0.25 |
| Acrylamide Triquat⁶ | 0.25 | 0 |
| Methyl Paraben | 0 | 0 |
| Sodium Benzoate | 0.25 | 0.25 |
| Salicylic Acid USP | 0 | 0 |
| Tetrasodium EDTA Tetrahydrate | 0.15 | 0.15 |
| Trisodium Ethylenediamine Disuccinate | 0.10 | 0.10 |
| Panthenol | 0.03 | |
| Panthenyl Ethyl Ether | 0.03 | |
| methylchloroisothiazolinone | 0.0005 | 0.0005 |

¹Stepanol SLS from Stepan
²P&G Chemicals
³Amphosol HCA-HP from Stepan
⁴Besil DM500 from Wacker
⁵N-Hance 3196 from Aqualon
⁶Mirapol AT-1 from Rhodia

### Comparative Conditioner Example A

| EXAMPLE | A |
|---|---|
| Stearyl alcohol ¹ | 2.3 |
| Behentrimonium methosulfate² | 1.2 |
| Cetyl alcohol³ | 0.9 |
| Bis-aminopropyl dimethicone⁴ | 1 |
| Fragrance | 0.5 |
| Benzyl alcohol | 0.4 |
| Dicetyldimonium chloride | 0.35 |
| Propylene glycol | 0.12 |
| Disodium edta | 0.1 |
| Panthenol | 0.03 |
| Panthenyl Ethyl Ether | 0.03 |
| Methylchloroisothiazolinone | 0.0005 |

¹ P&G Chemical
²Feixiang Chemicals Zhangjiagang
³P&G Chemicals
⁴Momentive

### Test Method

A. In Home Product Use Test Method

Naïve female panelists in the U.S., ranging in age from 18-65 participated in this usage study. The study was executed and data was collected by an independent marketing research supplier. Research panelists were voluntary participants from the supplier's on-line database and represented a spectrum of ethnicities, hair types and lengths, income levels, household sizes and geographic regions within the U.S. on-line population. The recruitment criteria included a minimum shampoo frequency of 3 times a week (or more) and usage of a rinse-off conditioner in at least half of the shampoo occasions. In addition to the above recruitment criteria, the supplier also ensured that upon placement every test leg was balanced across a spectrum of hair lengths.

Panelists were given a test shampoo & rinse-off conditioner to use in place of their typical products for four weeks. Throughout the study period, the women followed their typical usage routine. Shampoo and conditioner dosage and frequency of use were determined by the panelists. The test products were placed in plain white packaging and simply labeled as “Shampoo” and “Rinse-off Conditioner” test products. Neither brand nor benefit context were provided. Throughout the study period, panelists were permitted to use their normal styling products and follow their typical drying and styling routine.

At the end of the study period, a self-administered, on-line survey was completed by each panelist. Study participants were asked to rate the test “Shampoo” and “Rinse-off Conditioner” combination on a standard 5 point “Poor to Excellent” scale, overall and for a series of Hair Care related benefits. The total base size of completed evaluations for each test “Shampoo” and “Rinse-off Conditioner” combination was a minimum of 200 women. In addition to the full panel, data was also analyzed among certain sub-populations seeking specific benefits. These sub-populations consisted of a minimum of 45 women. For this analysis, panelists seeking smoothing benefits from their shampoo and rinse-off conditioner were studied. This population was selected because smooth feel of hair in both wet and dry states are important indications of a smooth end result. All data was then analyzed using standard statistical tests at 90% Confidence and 80% Power.

### Shampoo Composition

Described herein is a hair care composition, such as a shampoo composition comprising: a a) surfactant; b) a cationic deposition polymer; and c) a silicone emulsion; d) carrier, as well as an e) an optional benefit agent. The shampoo and conditioner combination (system) delivers substantially better wet and dry smooth feel as determined by a blinded consumer usage test among panelists who are seeking a smoothing benefit (Table 1). These unique shampoo and conditioner systems (Shampoo Examples 1 and 7 and Conditioner Example 1) demonstrate better smooth feel of the hair wet and dry than the same shampoo paired with a different conditioner (Shampoo Example 1 and Comparative Conditioner Example A) and the same conditioner paired with different shampoos (Comparative Shampoo Examples A, and B, and Conditioner Example 1). Panelists' assessment of these benefits was completed following four weeks of using the designated shampoo and conditioner pairing under normal usage conditions. See Table 1.

TABLE 1
Average Ratings for Smooth Feel of Wet and Dry Hair

| | Among Smooth Seeking Females, 18-65 Shampoo Rinse-Off Conditioner Users | | | | | |
|---|---|---|---|---|---|---|
| | | Shampoo Example | Comparative | Comparative | | |
| | Shampoo Example | 1/Comparative | Shampoo Example | Shampoo Example | Shampoo Example | |
| | 1/Conditioner | Conditioner | A/Conditioner | B/Conditioner | 7/Conditioner | Std. |
| | Example 1 | Example A | Example 1 | Example 1 | Example 1 | Dev. |
| Base Size | (63) | (46) | (60) | (50) | (48) | |
| 5 pt. Rating Scale: | | | | | | |
| 0 = Poor to | | | | | | |
| 100 = Excellent | | | | | | |
| Avg. Rating | 85bcd | 68 | 66 | 74 | 77c | 5.74 |
| for Wet Hair | | | | | | |
| Feeling Smooth | | | | | | |
| after System | | | | | | |
| Avg. Rating | 77bcd | 61 | 58 | 66 | 71bc | 5.12 |
| for Dry Hair | | | | | | |
| Feeling Smooth | | | | | | |
| after System | | | | | | |

What is claimed is:

1) A method of providing a smooth feel to hair comprising:
a. applying to the hair a shampoo composition comprising:
i. from about 2% to about 50% of one or more detersive surfactants, by weight of the shampoo composition;
ii. from about 0.1 to about 0.25 by weight, of a cationic guar deposition polymer having a molecular weight of from about 100,000 g/mol to about 2.2 million g/mol;
iii. from about 0.1 to about 1.75 by weight, of a silicone emulsion comprising an insoluble polysiloxane having a general formula of R¹—[O—SiR₂]—OR¹, wherein n is an integer, R is a substituted or unsubstituted C₁ to C₁₀ alkyl or aryl, and R¹ is a hydrogen or a substituted or unsubstituted C₁ to C₁₀ alkyl or aryl, wherein the insoluble polysiloxane has a molecular weight within the range from about 50,000 to about 500,000 g/mol, and an average particle size within the range from about 30 nm to about 10 μm, wherein a total content of a cyclic polysiloxane having a general formula: 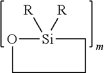
is present in the silicone emulsion in an amount less than about 2.5 wt % based on the total weight of the insoluble polysiloxane and the cyclic polysiloxane, wherein R is as defined above, wherein m is 4 or 5,
iv. from about 20% to about 95% of a first aqueous carrier, by weight of the shampoo composition;
b. rinsing the shampoo composition from the hair;
c. applying to the hair a conditioner composition comprising:
i. a conditioner gel matrix comprising:
a) from about 0.1% to about 20% of one or more high melting point fatty compounds, by weight of the gel matrix;
b) from about 0.1% to about 10% of a cationic surfactant, by weight of the gel matrix; wherein the cationic surfactant system comprises a mono-long alkyl quaternized ammonium salt
c) at least 20% of a second aqueous carrier, by weight of the gel matrix;
d) silicone conditioning agents
d. rinsing the conditioner composition from the hair.

2) The method of claim 1, wherein the silicone conditioning agent of the hair conditioner is an aminosilicone.

3) The method of claim 1, wherein the deposition polymer comprises a blend of cationic deposition polymers.

4) The method of claim 3, wherein the deposition polymers comprises a first cationic guar polymer having a molecular weight of from about 500,000 g/mol to about 700,000 g/mol and a second cationic guar polymer having a molecular weight of from about 1,000,000 g/mol to about 2,000,000 g/mol.

5) The method of claim 1, wherein the shampoo composition further comprises one or more additional benefit agents.

6) The method of claim 5, wherein the one or more additional benefit agents is an anti-dandruff agent.

7) The method of claim 1, wherein the cationic guar deposition polymer has a molecular weight of about 1.7 million g/mol.

8) The method of claim 1 wherein the mono-long alkyl quaternized ammonium salt cationic surfactant is behentrimonium chloride

9) The method of claim 1, wherein the shampoo composition has a pH of from about 3.5 to about 7.5.

10) The method of claim 1, wherein the first aqueous carrier is water.

11) The method of claim 1, wherein the conditioner composition further comprises one or more additional benefit agents.

12) The method of claim 1, wherein R is methyl and R¹ is hydrogen.

13) The method of claim 1, wherein the silicone emulsion has m of 4 and the total content of a cyclic polysiloxane is less than about 1.5 wt %.

14) The method of claim 1, wherein the silicone emulsion has m of 4 and the total content of a cyclic polysiloxane is less than about 1.0 wt %.

15) The method of claim 1, wherein the silicone emulsion has a viscosity up to about 500,000 cPs.

16) The method of claim 15, wherein the silicone emulsion has a viscosity within the range from about 100,000 cPs to about 200,000 cPs.

17) The method of claim 1, wherein the insoluble polysiloxane has an average particle size (D₅₀) within the range from about 50 nm to about 150 nm.
